(19) United States
(12) Patent Application Publication
(10) Pub. No.: US 2021/0217484 A1
(43) Pub. Date: Jul. 15, 2021

(54) VARIATIONAL AUTOENCODER FOR BIOLOGICAL SEQUENCE GENERATION

(71) Applicant: ModernaTX, Inc., Cambridge, MA (US)

(72) Inventors: Andrew Giessel, Cambridge, MA (US); Athanasios Dousis, Cambridge, MA (US); Iain McFadyen, Arlington, MA (US)

(73) Assignee: ModernaTX, Inc., Cambridge, MA (US)

(21) Appl. No.: 17/145,164

(22) Filed: Jan. 8, 2021

Related U.S. Application Data
(60) Provisional application No. 62/959,406, filed on Jan. 10, 2020.

(51) Int. Cl. G16B 5/20; G16B 20/20; G16B 40/00

(52) U.S. Cl. CPC G16B 5/20; G16B 40/00; G16B 20/20

(57) ABSTRACT

Techniques for manufacturing a variant of a target protein. The techniques may include accessing a latent variable statistical model (LVSM) configured to generate output indicating one or more biological sequences corresponding to one or more variants of the target protein and using the LVSM to generate a first output indicating a first biological sequence associated with a first variant of the target protein. The techniques further include manufacturing, using the first biological sequence, a first biological molecule to produce the first variant of the target protein.

### RELATED APPLICATIONS

This application claims the benefit under 35 U.S.C. § 119(e) of U.S. Provisional Patent Application Ser. No. 62/959,406, filed Jan. 10, 2020, titled “VARIATIONAL AUTOENCODER FOR BIOLOGICAL SEQUENCE GENERATION”, the entire contents of which are incorporated by reference herein.

### FIELD

Aspects of the technology described herein relate to constructing and using statistical models for generating biological sequences, including those associated with protein variants, to manufacture as biological molecules. In particular, some aspects of the technology described herein relate to determining a biological sequence associated with a variant of a protein of interest, including an amino acid sequence of the variant and a nucleotide sequence that encodes for the variant.

### BACKGROUND

Advances in engineering novel biological molecules, such as nucleic acids and proteins, have allowed for the implementation of non-naturally occurring biological molecules in many areas of biotechnology and medicine. These new biological molecules may have one or more enhanced characteristics (e.g., stability, expression level, specificity) in comparison to their wildtype versions. In turn, the enhanced characteristics of the biological molecules may promote their use in various current applications and allow for the further development of applications where biological molecules are utilized.

Bioprocessing applications involve using engineered biological molecules to produce particular products, including drugs, biofuels, chemicals, and food. These bioprocessing applications may benefit from engineering the biological molecules to improve certain characteristics such as robustness, specificity and reproducibility of the bioprocessing production. For example, a DNA polymerase needed for a particular bioprocessing application conducted at specific environmental conditions (e.g., high heat) may be engineered to have a desired stability under those environmental conditions to allow for the synthesis of nucleic acids, whereas the wildtype version of the DNA polymerase would not function or have limited function in such an environment.

In medicine, there is widespread interest is developing the use of biological molecules as possible therapies and treatments for specific medical conditions and diseases. Such biological therapeutic products include protein- and nucleic acid-based drugs. The development and manufacture of such biological therapeutic products may involve engineering the biological molecule to have particular characteristics and/or functionality specific to the medical condition or disease being treated.

### SUMMARY

Some embodiments are directed to a method of manufacturing a variant of a target protein, comprising: accessing a latent variable statistical model (LVSM) configured to generate output indicating one or more biological sequences corresponding to one or more variants of the target protein; using the LVSM to generate a first output indicating a first biological sequence associated with a first variant of the target protein; and manufacturing, using the first biological sequence, a first biological molecule to produce the first variant of the target protein.

In some embodiments, the first variant of the target protein has at least the same activity as the target protein. In some embodiments, the first variant of the target protein has enhanced activity in comparison to the target protein.

In some embodiments, the target protein is a human protein, and manufacturing the first biological molecule further comprises synthesizing the first biological molecule for administration to a human subject. In some embodiments, the method further comprises administering a treatment comprising the first biological molecule to the human subject.

In some embodiments, the LVSM was trained using biological sequences including a human biological sequence corresponding to the human protein. In some embodiments, the biological sequences further include biological sequences corresponding to the target protein occurring in organisms other than a human. In some embodiments, the biological sequences correspond to proteins having substantially similar functions in different species. In some embodiments, training the LVSM comprises aligning the biological sequences and using the aligned biological sequences to train the LVSM.

In some embodiments, the first variant has at least 30 residues having a different amino acid than the target protein. In some embodiments, the first variant has at least 5 residues having a different amino acid than the target protein. In some embodiments, the first variant has at least 95% sequence similarity with the target protein for at least one conserved region.

In some embodiments, a surface site of the first variant has a different amino acid than the target protein. In some embodiments, a core site of the first variant has a different amino acid than the target protein. In some embodiments, a boundary site of the first variant has a different amino acid than the target protein.

In some embodiments, the first biological molecule includes a nucleotide sequence that encodes for the first variant. In some embodiments, the first biological molecule is a messenger ribonucleic acid (mRNA). In some embodiments, the first biological molecule is a deoxyribonucleic acid (DNA).

In some embodiments, manufacturing the first biological molecule further comprises using the first biological molecule to synthesize the first variant of the target protein. In some embodiments, the first biological molecule is the first variant of the target protein.

In some embodiments, using the LVSM further comprises: identifying parameters of a distribution over a latent space of the LVSM corresponding to an input biological sequence obtained at least in part by sequencing a biological sample of a human; identifying, using the parameters, a point in the latent space of the LVSM; and identifying, using the point and the LVSM, the first biological sequence associated with the first variant of the target protein.

In some embodiments, the first output generated from the LVSM indicates a plurality of biological sequences associated with a respective plurality of variants of the target protein including the first variant, and the method further comprises: determining a characteristic for each of the plurality of variants; and selecting, from among the plurality of biological sequences, the first biological sequence based on the characteristic. In some embodiments, the protein characteristic is selected from the group consisting of protein expression level, protein half-life, protein subcellular localization, protein tissue specificity, protein immunogenicity, and protein cofactor-dependence specificity.

In some embodiments, the LVSM includes a multi-layer neural network. In some embodiments, the LVSM includes a neural network having one or more convolutional layers. In some embodiments, the LVSM includes a variational autoencoder.

Some embodiments are directed to a system comprising: at least one hardware processor; and at least one non-transitory computer-readable storage medium storing processor-executable instructions that, when executed by the at least one hardware processor, cause the at least one hardware processor to perform a method. The method comprises accessing a latent variable statistical model (LVSM) configured to generate output indicating one or more biological sequences corresponding to one or more variants of a target protein; using the LVSM to generate a first output indicating a first biological sequence associated with a first variant of the target protein; and manufacturing, using the first biological sequence, a first biological molecule to produce the first variant of the target protein.

Some embodiments are directed to at least one non-transitory computer-readable storage medium storing processor-executable instructions that, when executed by at least one hardware processor, cause the at least one hardware processor to perform: accessing a latent variable statistical model (LVSM) configured to generate output indicating one or more biological sequences corresponding to one or more variants of a target protein; using the LVSM to generate a first output indicating a first biological sequence associated with a first variant of the target protein; and manufacturing, using the first biological sequence, a first biological molecule to produce the first variant of the target protein.

Some embodiments are directed to a method of determining a variant of a target protein, comprising: identifying, for a latent variable statistical model (LVSM) configured to generate output indicating one or more biological sequences corresponding to one or more variants of the target protein, parameters of a distribution over a latent space of the LVSM corresponding to an input biological sequence obtained at least in part by sequencing a biological sample of a human; identifying, using the parameters, a point in the latent space of the LVSM; and identifying, using the point and the LVSM, a first output biological sequence associated with a first variant of the target protein.

In some embodiments, identifying the point comprises: sampling the point from the latent space according to the distribution. In some embodiments, identifying the point comprises: scaling the distribution, at least in part, by modifying the parameters to obtain a scaled distribution; and sampling the point from the latent space according to the scaled distribution. In some embodiments, identifying the point comprises sampling the point using a concentric sampling technique. In some embodiments, identifying the point comprises sampling the point using a random sampling technique. In some embodiments, identifying the point comprises sampling the point using an interpolation sampling technique. In some embodiments, identifying the point comprises sampling the point using a learned manifold sampling technique.

In some embodiments, the method further comprises identifying the parameters of the distribution by providing the input biological sequence as input to the LVSM.

In some embodiments, the LVSM is trained using biological sequences corresponding to proteins occurring in different types of organisms. In some embodiments, the biological sequences include a human biological sequence. In some embodiments, the biological sequences correspond to proteins having substantially similar functions in different species.

In some embodiments, the method further comprises identifying a second point using the parameters; and identifying, using the second point and the LVSM, a second output biological sequence corresponding to a second variant of the target protein different from the first variant.

In some embodiments, the LVSM includes a multi-layer neural network. In some embodiments, the LVSM includes a neural network having one or more convolutional layers. In some embodiments, the LVSM includes a variational autoencoder. In some embodiments, the LVSM comprises an encoder portion and a decoder portion. In some embodiments, the encoder portion is configured to map input biological sequences to distributions over the latent space of the LVSM. In some embodiments, the decoder portion is configured to map individual points in the latent space of the LVSM to respective output indicating a respective biological sequence corresponding to a variant of the target protein.

In some embodiments, the method further comprises manufacturing, using the output biological sequence, a first biological molecule to produce the first variant of the target protein. In some embodiments, the target protein is a human protein, and manufacturing the first biological molecule further comprises synthesizing the first biological molecule for administration to a human subject. In some embodiments, the method further comprises administering a treatment comprising the first biological molecule to the human subject.

In some embodiments, the first variant has at least 30 residues having a different amino acid than the target protein. In some embodiments, the first variant has at least 5 residues having a different amino acid than the target protein. In some embodiments, the first variant has at least 95% sequence similarity with the target protein for at least one conserved region.

Some embodiments are directed to at least one non-transitory computer-readable storage medium storing processor-executable instructions that, when executed by at least one hardware processor, cause the at least one hardware processor to perform: identifying, for a latent variable statistical model (LVSM) configured to generate output indicating one or more biological sequences corresponding to one or more variants of the target protein, parameters of a distribution over a latent space of the LVSM corresponding to an input biological sequence obtained at least in part by sequencing a biological sample of a human; identifying, using the parameters, a point in the latent space of the LVSM; and identifying, using the point and the LVSM, a first output biological sequence associated with a first variant of the target protein.

Some embodiments are directed to a system comprising: at least one hardware processor; and at least one non-transitory computer-readable storage medium storing processor-executable instructions that, when executed by the at least one hardware processor, cause the at least one hardware processor to perform a method. The method comprises identifying, for a latent variable statistical model (LVSM) configured to generate output indicating one or more biological sequences corresponding to one or more variants of the target protein, parameters of a distribution over a latent space of the LVSM corresponding to an input biological sequence obtained at least in part by sequencing a biological sample of a human; identifying, using the parameters, a point in the latent space of the LVSM; and identifying, using the point and the LVSM, a first output biological sequence associated with a first variant of the target protein.

### BRIEF DESCRIPTION OF DRAWINGS

Various aspects and embodiments will be described with reference to the following figures. The figures are not necessarily drawn to scale.

FIG. 1 is a diagram of an illustrative process for generating and using a latent variable statistical model (LVSM) to output biological sequence(s) and manufacture biological molecule(s), using the technology described herein.

FIG. 2 is a schematic of a variational autoencoder (VAE) used for generating biological sequences, using the technology described herein.

FIG. 3 is a schematic of the latent space of a trained VAE used for generating biological sequences, using the technology described herein.

FIG. 4 is exemplary aligned training data used for training a LVSM, using the technology described herein.

FIG. 5 is a schematic illustrating sampling of the latent space of the trained VAE shown in FIG. 3 to generate output biological sequences, using the technology described herein.

FIG. 6A-6D are schematics illustrating different techniques for sampling a latent space of a LVSM, using the technology described herein.

FIG. 7A is a plot illustrating relative entropy obtained from training sequence data, using the technology described herein.

FIG. 7B is a plot illustrating relative entropy obtained from biological sequences generated from a trained LVSM, using the technology described herein.

FIG. 7C is a plot of the relative entropy shown in FIG. 7B versus the relative entropy shown in FIG. 7A.

FIG. 8A is a plot illustrating mutual information obtained from training sequence data, using the technology described herein.

FIG. 8B is a plot illustrating mutual information obtained from biological sequences generated from a trained LVSM, using the technology described herein.

FIG. 8C is a plot of the mutual information shown in FIG. 8B versus the mutual information shown in FIG. 8A.

FIG. 9A is a plot of total correlation for randomly generated biological sequences versus biological sequences used as training data, using the technology described herein.

FIG. 9B is a plot of total correlation for position conserved biological sequences versus biological sequences used as training data, using the technology described herein.

FIG. 9C is a plot of total correlation for biological sequences generated using a variational autoencoder, using the technology described herein.

FIG. 9D is a plot of sequence count versus reconstruction loss for the training sequences, VAE generated sequences, position conserved sequences, and randomly generated sequences, using the technology described herein.

FIG. 10 is a flow chart of an illustrative process for manufacturing a variant of a protein, using the technology described herein.

FIG. 11 is a flow chart of an illustrative process for determining a variant of a protein, using the technology described herein.

FIG. 12 is a block diagram of an illustrative computer system in which the technology described herein may be implemented.

### DETAILED DESCRIPTION

The inventors have recognized that various challenges can arise during engineering new biological molecules, such as proteins and nucleic acids (e.g., messenger RNA (mRNA)), particularly because of the high number of possible combinations of nucleoside and amino acid residues (subunits) that can form biological sequences, and the limited understanding of how changes to specific positions in a biological sequence impact overall functionality of a resulting biological molecule associated with the biological sequence. For example, in the context of protein engineering, there are 20 possible amino acids that could be located at each residue site and considering the impact of possible mutations to an existing amino acid sequence becomes more complex as the number of mutations grows because the number of amino acid combinations increases exponentially with the number of mutations. In addition, a protein may have critical residue sites which, if mutated, may impact the structural and/or functional integrity of the protein. A protein may also have residue sites that compensate for amino acid substitutions at other residues, diminishing or otherwise altering the effect of those amino acid substitutions. These additional relationships between protein structure and functionality can lead to further challenges when engineering new proteins, particularly if such relationships are generally unknown.

The inventors have recognized that conventional techniques for generating new functional biological macromolecules and for manufacturing biological molecules are limited in both their ability to: (1) consider a variety of possible substitutions of subunits (e.g., amino acids, nucleosides) within biological sequences; and (2) select biological sequences that can be manufactured. In particular, some conventional techniques may engineer biological sequences by restricting the location and number of mutations made in comparison with wildtype to maintain the overall structural integrity of a biological molecule having the biological sequence. This substantially limits the scope of which biological sequences are considered for a particular application and, thus, inhibits development of biological molecules for that application. Additionally, some conventional techniques may identify many possible biological sequences, but only some of those sequences may be functional as biological molecules, in large part because it may not be possible to predict the impact of certain substitutions on a biological molecule's secondary and tertiary structures.

In protein engineering, proper protein folding still involves many unknown factors, and thus it can be difficult to know which residues can be modified in an amino acid sequence and still lead to a properly folded protein. For example, some conventional techniques for engineering proteins involve using physics-based energy models, including molecular dynamics simulations and quantum mechanical simulations, to relate protein sequence information to protein structure as part of designing novel proteins that have particular functions. These techniques may be referred to as “rational protein design,” which uses the relationship between protein function and structure to design new proteins. Generally, these approaches involve using a known biological sequence for a naturally-occurring protein and sequentially making one mutation at a time to evaluate the impact of each individual mutation on the resulting protein structure. This systematic approach to designing novel proteins is generally used because of the lack of information relating to protein structure (e.g., crystal structure of a protein of interest), and thus, it is challenging to determine the impact specific mutations may have on the variant protein's structure. Generally, evaluating each subsequent mutation involves synthesizing a protein having that mutation (and any other preceding mutations) and, if the protein is correctly folded, assessing the characteristics of the folded protein. Additionally, there are significant computational challenges associated with the energy models used in rational protein design, particularly as the number of mutations being simultaneously considered increases.

In addition, some conventional techniques for engineering proteins may involve using a natural selection process for proteins, or the genes that encode for proteins, by subjecting a gene to iterative cycles of mutations to create a variant library, selecting some of those variants as having a desired function, and amplifying the selected variants to generate templates for the subsequent iteration. This process may be referred to as “directed evolution” because it mimics the evolutionary process in a laboratory setting with the goal of generating a variant protein having particular characteristics. Such techniques tend to lack any computational component for determining the mutations because, generally, the mutations originate through biological laboratory processes, including random point mutations (e.g., using error-prone polymerase chain reaction (PCR)), insertions, deletions, and gene recombination. Since the mutations are generally arbitrarily made, it is a challenge to use such directed evolutionary techniques to systematically explore possible mutations that lead to variants having desired characteristics. In addition, these approaches are time consuming and expensive because of the costs associated with synthesizing and assessing proteins at each stage of development to evaluate the impact mutations have on the protein's overall structure and function.

These conventional techniques are limited in the variety of variants generated, both in terms of the types and locations of mutations, as well as in the time and costs associated with generating a single variant. In turn, these limitations impact technological progress in applications where novel biological molecules, including engineered proteins, may be utilized. In the context of bioprocessing, the inability to efficiently and inexpensively manufacture biological molecules limits the extent to which biological molecules are used in industrial and pharmaceutical processes. In addition, these limitations impact the ability to expedite production of new drugs for both treating certain medical conditions and personalizing treatments for different patients. In the context of personalized medicine, the ability to efficiently and inexpensively develop new biological molecules for different patients becomes particularly important in having these types of treatments become more widely available.

To address some of the aforementioned problems with conventional techniques for manufacturing biological molecule (e.g., protein) variants, the inventors have developed improved biological sequence engineering techniques. The improved techniques allow for generating variant biological sequences having a greater variety of mutations, both in terms of location and number, in comparison to conventional biological sequence engineering approaches. The techniques developed by the inventors do not rely, in some embodiments, on any available explicit protein structure information in determining these new variants. Rather, in some embodiments, the techniques developed by the inventors use known biological sequences across multiple species, which are more readily available than protein structure information in any case, to learn a statistical model for generating biological sequence variants. In some embodiments, the statistical model may be a latent variable statistical model (LVSM) (e.g., a variational autoencoder) having a latent space generated during the training process and representative of relationships between features of biological sequences used as training data. The output biological sequences are generated by sampling from the latent space.

Some genes and their corresponding proteins are highly conserved across different types of organisms, including different species (e.g., human, bacteria) and/or individuals of the same species that have different genomes. In this context, highly conserved sequence regions are identical or substantially similar biological sequences and may give rise to proteins having similar functions. The inventors have further recognized that these highly conserved biological sequences can be implemented in determining protein variants and their corresponding biological sequences. Accordingly, some embodiments of the technology described herein are directed to techniques that involve using biological sequences corresponding to a target protein occurring in different types of organisms to train a LVSM. To generate novel biological sequences associated with variants of the target protein occurring in humans using the trained LVSM, the latent space of the LVSM may be sampled using a distribution over the latent space whose parameters correspond to the human biological sequence, and the sampled point may be used to generate a corresponding output sequence (e.g., by using a decoder portion of the LVSM). In this way, these techniques developed by the inventors for determining biological molecules may allow for evolutionary conserved regions of the target protein across different types of organisms to be considered in generating a biological sequence associated with a variant of the target protein occurring in a human.

The biological sequences generated by using the techniques developed by the inventors have particular advantages relative to biological sequences obtained using conventional protein engineering techniques. In some instances, the generated biological sequences may account for relationships between different protein regions that impact overall protein functionality such that the effect of compensatory regions within a protein is limited. As a result, a variant of the target protein produced using a biological sequence generated using the techniques described herein may have enhanced activity, or at least the same activity, as a wildtype version of the target protein. In addition, these techniques developed by the inventors may generate biological sequences that are more likely to be successfully manufactured as biological molecules, including nucleic acids and proteins, in comparison to conventional protein engineering techniques. According to some aspects, successful manufacturing of a biological molecule may involve successful synthesis of a biological molecule having a generated biological sequence. In the context of manufacturing a protein, successful manufacturing may include accurate transcription of an mRNA molecule to an amino acid molecule and correct folding of the amino acid molecule into a protein, where the resulting protein has a desired functionality.

Some embodiments described herein address all of the above-described issues that the inventors have recognized with determining biological sequences and manufacturing biological molecules. However, not every embodiment described herein addresses every one of these issues, and some embodiments may not address any of them. As such, it should be appreciated that embodiments of the technology described herein are not limited to addressing all or any of the above-described issues with determining biological sequences and manufacturing biological molecules.

Some embodiments involve accessing a latent variable statistical model (LVSM) configured to generate output indicating one or more biological sequences corresponding to one or more variants of a protein, and using the LVSM to generate an output indicating a biological sequence associated with a variant of the target protein. The architecture of the LVSM may include a multi-layer neural network and a neural network having one or more convolutional layers. In some embodiments, the LVSM is a variational autoencoder. In such embodiments, the LVSM may include an encoder portion and a decoder portion. The encoder portion may be configured to map input biological sequences to parameters of distributions over the latent space of the LVSM. The decoder portion may be configured to map individual points in the latent space of the LVSM to respective output indicating a respective biological sequence corresponding to a variant of the target protein.

The biological sequence may be used to manufacture a biological molecule to produce the variant of the target protein. In some embodiments, the variant may have the same or substantially similar activity as the target protein. In some embodiments, the variant may have enhanced activity in comparison to the target protein. For example, in the context of engineering an enzymatic protein it may be desirable that the variant of the target protein have at least the same, and possibly enhanced, enzymatic activity in comparison to the known target enzyme.

Some embodiments involve techniques for training the LVSM to configure the LVSM to generate output indicating one or more biological sequences corresponding to one or more variants of a target protein. In some embodiments, training the LVSM may involve using multiple biological sequences, including a human biological sequence corresponding to the human target protein. The biological sequences may include biological sequences corresponding to the target protein occurring in organisms other than a human. In some embodiments, the biological sequences may correspond to proteins having substantially similar functions in different species, which may include species other than human. The biological sequences may include highly conserved regions, such as particular nucleotide positions or amino acid residues, across different types of organisms, including different species (e.g., human, bacteria) and/or different genomes within the same species. In some aspects, certain regions of the biological sequences may be considered as being “highly conserved” when those regions have identical amino acids at particular residues, and a percentage of identical residues may be considered as “sequence identity.” In some embodiments, the biological sequences may correspond to proteins having conserved regions with a high sequence identity, such as a sequence identity that is of at least 95%, 90%, 80%, or 70%, among the biological sequences for a particular conserved region. In contrast, the biological sequences overall may have a particularly low sequence identity, such as in the range of 40-50%. According to some embodiments, the biological sequences may correspond to proteins having substantially similar function(s) within different species. Regions of the biological sequences may be considered as being “highly conserved” when those regions have similar physiochemical properties, which may include both regions where the same amino acid is at one or more residues and regions where the amino acid differs at a residue, but the different residues have similar properties. A percentage of residues with similar physicochemical properties may be considered as “sequence similarity.” In some embodiments, the biological sequences may correspond to proteins having conserved regions where the sequences have a high sequence similarity, such as at least 95%, 90%, 80%, or 70% sequence similarity among the biological sequences for a particular conserved region. The biological sequences may be processed prior to using them to train the LVSM. In some embodiments, training the LVSM comprises aligning the biological sequences and using the aligned biological sequences to train the LVSM.

Some embodiments involve techniques for sampling the trained LVSM by using an input biological sequence obtained by sequencing a biological sample of a human. The biological sequence may correspond to the target protein, such as an amino acid sequence of the target protein or a nucleotide sequence (e.g., RNA) that encodes for the amino acid sequence of the target protein. In some embodiments, determining a variant of the target protein may involve identifying, for the LVSM, parameters (e.g., means, variances, higher-order moments, etc.) of a distribution over a latent space of the LVSM corresponding to the input biological sequence by providing the input biological sequence as input to the LVSM. Determining the variant of the target protein may further include using the parameters to identify a point in the latent space of the LVSM (e.g., by sampling the point from a distribution over the latent space of the LVSM defined by the parameters) and using the point to generate an output biological sequence associated with a variant of the target protein. Additional biological sequences corresponding to variants of the target protein different than the first variant may be determined by identifying additional points in the latent space of the LVSM (e.g., by drawing additional samples in the latent space in accordance with the distribution specified by the parameters). Accordingly, some embodiments involve identifying a second point using the parameters (e.g., by drawing a sample from the distribution defined by the parameters), and generating, using the second point and the LVSM, a second output biological sequence corresponding to a second variant of the target protein different than the first variant.

In some embodiments, determining a variant of the target protein may involve identifying, for the LVSM, a first point in a latent space of the LVSM corresponding to the input biological sequence by providing the input biological sequence as an input to the LVSM. In some aspects, the first point may correspond to a mean for a distribution generated by inputting the input biological sequence to the LVSM. Determining the variant of the target protein may further include using the first point to identify a second point in the latent space of the LVSM and using the second point to generate an output biological sequence associated with a variant of the target protein. Additional biological sequences corresponding to variants of the target protein different than the first variant may be determined by identifying additional points using the first point and the LVSM. Accordingly, some embodiments involve identifying a third point using the first point, and generating, using the third point and the LVSM, a second output biological sequence corresponding to a second variant of the target protein different than the first variant.

Various sampling techniques may be implemented to identify point(s) in the latent space that are used for generating biological sequence(s) associated with variant(s) of the target protein. Some embodiments involve identifying parameters of a distribution corresponding to an input biological sequence and using the parameters to identify a point in the latent space. In such embodiments, identifying the point may include sampling the point from the latent space according to the distribution. In some embodiments, identifying the point may include scaling the distribution, at least in part, by modifying the parameters to obtain a scaled distribution (e.g., when the parameters involve variances, modifying the parameters may involve scaling the variances by one or more scaling factors), and sampling the point from the latent space according to the scaled distribution.

Some embodiments involve identifying a first point in the latent space correspond to an input biological sequence and using the first point to identify a second point in the latent space, where the second point is used to determine a variant of a target protein. In some embodiments, identifying the second point may include identifying a region of the latent space containing the first point and sampling the second point from the region. The region of the latent space may be within a threshold distance of the first point. In embodiments where the first point corresponds to the biological sequence of the human protein, sampling in the region containing the first point may be considered as sampling near the human biological sequence. Additional sampling techniques that may be used in identifying the second point include concentric sampling techniques, random sampling techniques, interpolation sampling techniques, and learned manifold sampling techniques.

According to some embodiments, an output generated from the LVSM may indicate multiple biological sequences associated with different variants of the target protein and techniques for selecting a particular variant may be based on one or more protein characteristics of the different variants. In some embodiments, the selection process may involve determining a characteristic for each of the plurality of variants, and selecting, from among the plurality of biological sequences, a particular biological sequence based on the identified characteristic. Examples of protein characteristics that may be used in selecting a biological sequence include protein expression level, protein half-life, protein subcellular localization, protein tissue specificity, protein immunogenicity, and protein cofactor-dependence specificity.

A variant protein outputted by the LVSM may differ from the target protein at one or more residues, which may be located at different sites of the protein. The number of residue sites having mutations where the variant protein has a different amino acid in comparison to the target protein may be in the range of 1-100 residues, or any number or range of numbers in that range. In embodiments where a distribution over the latent space corresponding to an input biological sequence is scaled, the parameters may be modified to obtain a scaled distribution such that sampling a point in the latent space according to the scaled distribution generates an output biological sequence having a number of mutations within a desired range in comparison to the target protein. For example, in some embodiments, parameters of the distribution may be modified to obtain a scaled distribution that generates output biological sequences having a number of mutations in the range of 7 to 11 mutations in comparison to the target protein. In some embodiments, the variant may have at least 30 residues that have a different amino acid than the target protein. In some embodiments, the variant may have at least 5 residues that have a different amino acid than the target protein. In some embodiments, the variant may have at least 95% sequence similarity with the target protein for at least one conserved region. Different residue sites where the variant protein may have one or more different amino acids than the target protein may include surface sites, core sites, and boundary sites of the protein. A surface site of a protein corresponds to a residue located on an outer region, or surface, of the folded protein. A core site of a protein corresponds to a residue located on an inner region, or core, of the folded protein. A boundary site of a protein corresponds to a residue located on a boundary of a domain of the folded protein.

The techniques described herein may be applied to the manufacture of different types of biological molecules, including nucleic acids and proteins, which are used to produce or may be one or more variants of a target protein. In some embodiments, a manufactured biological molecule is a variant of the target protein. In some embodiments, a manufactured biological molecule may include a nucleotide sequence that encodes for a variant of the target protein. The biological molecule may be a nucleic acid, including deoxyribonucleic acid (DNA), ribonucleic acid (RNA), including different types of RNA, such as messenger RNA (mRNA). For example, the biological molecule may be an mRNA molecule and the variant of the target protein may be produced by translation of the mRNA using a ribosome. As another example, the biological molecule may be a DNA molecule, and the variant of the target protein may be produced by transcription of the DNA to an RNA molecule using RNA polymerase followed by subsequent translation.

In some embodiments where the target protein is a human protein, manufacturing the biological molecule may involve synthesizing the biological molecule for administration to a human subject. Some embodiments may further involve techniques for administering a treatment that includes the biological molecule to a human subject. For example, some embodiments may involve administering mRNA that encodes a variant of the target protein to a human and the human's cellular machinery, including their ribosomes, may be used in producing the variant of the target protein within the human's cells.

It should be appreciated that the various aspects and embodiments described herein be used individually, all together, or in any combination of two or more, as the technology described herein is not limited in this respect.

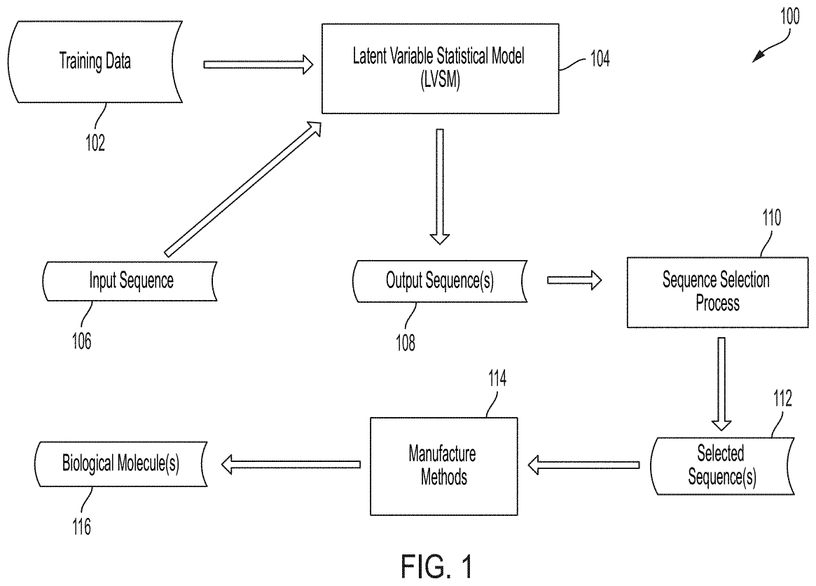

FIG. 1 is a diagram of an illustrative processing pipeline 100 for manufacturing a variant of a protein, which may include accessing a latent variable statistical model (LVSM) configured to generate output indicating one or more biological sequences corresponding to one or more variants of a protein, and using the LVSM to generate an output indicating a biological sequence associated with a variant of the target protein, in accordance with some embodiments of the technology described herein.

As shown in FIG. 1, LVSM 104 may be accessed to generate output sequence(s) 108, which may correspond to one or more variants of a target protein. In particular, input biological sequence 106 may be used as an input to the LVSM 104 to generate output sequence(s) 108. LVSM 104 may have any suitable architecture, including a multi-layer neural network and a neural network having one or more convolutional layers. In some embodiments, LVSM 104 is a variational autoencoder (VAE). In such embodiments, LVSM 104 includes an encoder portion and a decoder portion. The encoder portion may be configured to map input biological sequences to distributions (e.g., to parameters of distributions) over the latent space of LVSM 104. In some embodiments, the encoder portion may be configured to map input biological sequences to points in the latent space of LVSM 104, where the points may correspond to means of the distributions. The decoder portion may be configured to map individual points in the latent space of LVSM 104 to respective output indicating a respective biological sequence corresponding to a variant of the target protein.

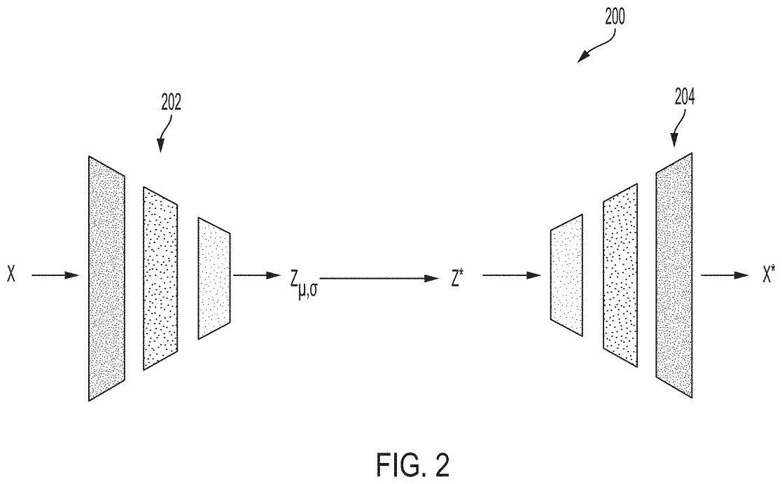

In some embodiments, the LVSM 104 may be implemented as a variational autoencoder (VAE), for example as a VAE having the architecture shown in FIG. 2. As shown in FIG. 2, VAE 200 includes encoder portion 202 and decoder portion 204. Encoder portion 202 is configured to map an input, X, into a distribution over a latent space of VAE 200. The distribution may have parameters, Z,_{μ,σ,}which may include mean(s) and variance(s). Each of the parameters, Z,_{μ, σ,} may include a mean, μ, and a variance, σ, of a respective distribution. The parameters, in turn, define a distribution over individual points in the latent space. In some embodiments, the distribution may be a multidimensional Gaussian distribution having any suitable number of dimensions, and parameters, Z,_{μ,σ,} may include means and variances associated with the different dimensions. Decoder portion 204 is configured to map individual points, Z*, in the latent space of VAE 200 to a respective output X*. A point in the latent space may be identified using parameters of a distribution over the latent space, and decoder portion 204 may map the point to an output. In some embodiments, VAE 200 may have a likelihood described using a Gaussian mixture model, with the statistical means and variances of the Gaussian mixture model specified by the parameters, Z,_{μ,σ,} . Additional examples of variational autoencoders which may be implemented as LVSM 104 are described in “Auto-Encoding Variational Bayes” by Diederik P. Kingma and Max Welling, Proceedings of the 2nd International Conference on Learning Representations (ICLR), 2013, which is incorporated herein by reference in its entirety.

In some embodiments, an encoder portion of a VAE may have one or more convolutional layers, one or more additional layers, including pooling layers (e.g., max pooling, average pooling), and one or more non-linear functions (e.g., rectified linear unit (ReLU), sigmoid). A decoder portion of the VAE may have one or more transpose convolutional layers, one or more additional layers, and one or more non-linear functions. The encoder portion and the decoder portion may have any suitable number of layers. As shown in FIG. 2, VAE 200 has a neural network architecture having an “hour-glass” configuration where encoder portion 202 has three convolutional layers with decreasing size and decoder portion 204 has three convolutional layers having increasing size. In some embodiments, the convolutional layers of encoder portion 202 and decoder portion 204 may have sizes of 128, 96, and 64 in combination with 3×3 filters. In such embodiments, the latent space may have a size of 64. Although VAE 200 shown in FIG. 2 has encoder portion 202 and decoder portion 204 having symmetric layers both in terms of number of layers and size of the layers, it should be appreciated that other VAE architectures may be implemented as LVSM 104, including architectures that are asymmetric in terms of number of layers and/or size of the layers.

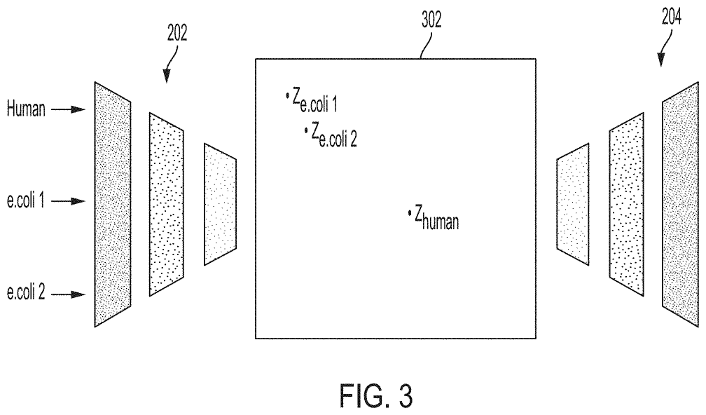

FIG. 3 is a schematic of latent space 302 of VAE 200 and illustrates how different biological sequences map to different points within latent space 302. In particular, the “Human” biological sequence maps to the Zₕᵤₘₐₙ point of latent space 302, the “e. coli 1” biological sequence maps to the Z_{e.coli 1} point of latent space 302, and the “e. coli 2” biological sequence maps to Z_{e.coli 2} point of latent space 302. As shown in FIG. 2, both e. coli biological sequences map to a region of latent space 302 where points Z_{e.coli 1} and Z_{e.coli 2} are in close proximity to one another in comparison to Zₕᵤₘₐₙ. In embodiments where encoder portion 202 maps input biological sequences to a distribution over latent space 302, the different points shown in FIG. 3 may be means for the distributions corresponding to the different biological sequences. Since latent space 302 has two dimensions, in this example, each point in latent space 302 may correspond to the two means of a two-dimensional distribution. In particular, Zₕᵤₘₐₙ point may correspond to the means for a distribution corresponding to the “Human” biological sequence, Z_{e.coli 1} point may correspond to means for a distribution corresponding to the “e. coli 1” biological sequence, and Z_{e.coli 2} point may correspond to means for a distribution correspond to the “e. coli 2 ” biological sequence. Although latent space 302 is shown as having two dimensions, this is merely to simplify illustration, and it should be appreciated that the techniques described herein may involve using a LVSM having a latent space with any suitable number of dimensions.

As shown in FIG. 1, some embodiments may involve training LVSM 104 using training data 102. Training LVSM 104 may involve training LVSM 104 such that LVSM 104 is configured to generate an output indicating one or more biological sequences corresponding to one or more variants of a target protein. Training data 102 may include biological sequences and training LVSM 104 may involve using the biological sequences to generate a trained LVSM 104, which may be used in generating output sequence(s) 108. In some embodiments, the biological sequences of training data 102 may include a human biological sequence corresponding to a human target protein. In some embodiments, the biological sequences of training data 102 may include biological sequences corresponding to the target protein occurring in organisms other than a human. The biological sequences may correspond to proteins having substantially similar functions in different species. The biological sequences may be highly conserved, or at least have highly conserved regions, across different types of organisms. The biological sequences may include sequences associated with different species (e.g., human, bacteria) and/or different genomes within the same species. In some embodiments, the biological sequences may correspond to proteins having substantially similar function(s) within different species. The biological sequences may correspond to proteins and include highly conserved regions having a sequence similarity of at least 95%, 90%, 80%, or 70% among the biological sequences. Training data 102 may include a number of biological sequences in the range of 100 to 100,000, or any value or range of values in that range.

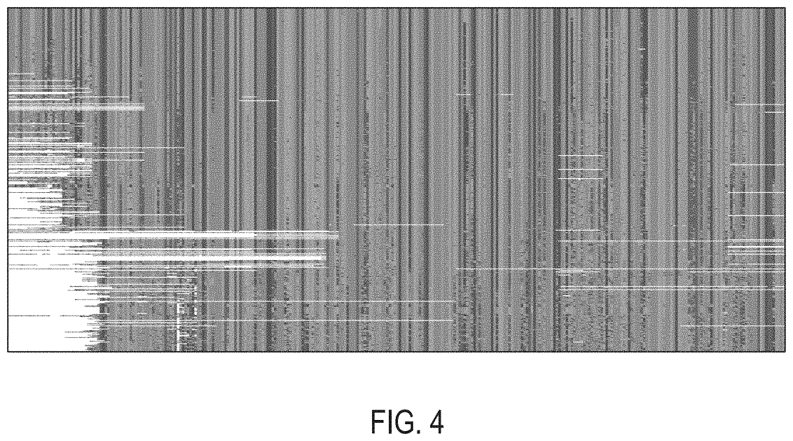

In some embodiments, training LVSM 104 comprises aligning biological sequences and using the aligned biological sequences to train LVSM 104. Aligning the biological sequences may involve aligning biological sequences to a reference sequence, which in some embodiments may be a human biological sequence. Sequence alignment techniques for aligning the biological sequences may include suitable multiple sequence alignment (MSA) software including Multiple Alignment using Fast Fourier Transform (MAFFT) and Multiple Sequence Comparison by Log-Expectation (MUSCLE). FIG. 4 is a plot of exemplary aligned training data illustrating the distribution of amino acids located at each residue site among a set of biological sequences used as training data 102 for LVSM 104. The grey shading shown in FIG. 4 corresponds to different types of amino acids. The horizonatal lines correspond to the different biological sequences. As shown by the aligned data in FIG. 4, some residue sites have the same amino acid across multiple biological sequences. Other residue sites have different amino acids across the multiple biological sequences.

Some embodiments may involve determining a set of biological sequences to be used in training LVSM 104 based on whether a particular biological sequence introduces a gap in aligning the sequences. For purposes of training LVSM 104, it may be desired to have the set of biological sequences used as training data to have few or no gaps at positions (e.g., an amino acid missing for a particular residue) in the aligned biological sequences. According to some embodiments, the set of biological sequences used in training may be determined such that no or few gaps are present in the alignment to a human biological sequence. Determining the set of biological sequences may involve filtering the biological sequences based on whether including a particular biological sequence in aligning the biological sequences introduces one or more gaps in the alignment. If a biological sequence is identified as introducing one or more gaps in the alignment, then the biological sequence may be excluded from the set of biological sequences used in training LVSM 104.

In some embodiments, filtering the biological sequences may involve aligning the biological sequences to generate a multiple sequence alignment and determining a gap score for each subunit position of the multiple sequence alignment (e.g., a column of the multiple sequence alignment, which may correspond to a particular residue), where the gap score depends on a number of gaps for its respective position. The gap scores may then be used in filtering the biological sequences to determine a set of biological sequences used for training. In some embodiments, the gap scores may be used to determine a sequence score for each biological sequence, and determining whether to include a particular biological sequence in the training data may depend on the value of the sequence score, such as if the sequence score is above a threshold value. Determining the sequence score for a particular biological sequence may include calculating the sequence score from the gap scores, such as by summing each gap score that corresponds to a gap in the biological sequence. In some embodiments, sequence length may be used in determining whether to include biological sequences in the training data. In some instances, biological sequences that are less than a certain length may be excluded from the training data. For example, biological sequences that have a length less than a percentage of the reference sequence (e.g., 80%) may be excluded from the training data.

According to some embodiments, using LVSM 104 to generate output sequence(s) 108 may involve using input sequence 106 to identify one or more points of the latent space to determine output sequence(s) 108. In particular, using LVSM 104 may involve identifying parameters of a distribution over the latent space of LVSM 104, and identifying, using the parameters, a point in the latent space. That point in turn may be used to generate an output sequence. Additional points in the latent space of LVSM 104 may be identified using the parameters, and those points may be used to generate additional output sequences. This process of identifying points in the latent space and their corresponding output sequences may be referred to as “sampling,” and it should be appreciated that different types of sampling techniques may be performed to generate output sequence(s). In the context of determining variants of a target protein using LVSM 104, input sequence 106 may include a biological sequence associated with the target protein (e.g., nucleotide sequence encoding for the target protein). Determining a variant of the target protein may involve identifying parameters (e.g., means, variances) of a distribution over the latent space of LVSM 104 corresponding to the biological sequence associated with the target protein, using the parameters to identify (e.g., sample) a point in the latent space. The point may be used to generate an output sequence. Additional points in the latent space of LVSM 104 may be identified using the parameters, and those points may be used to generate additional output sequences.

In some embodiments, using LVSM 104 may involve identifying a first point in the latent space of LVSM 104 and identifying, using the first point, a second point in the latent space. The second point may be used to generate an output sequence. Additional points in the latent space of LVSM 104 may be identified using the first point, and those points may be used to generate additional output sequences. In the context of determining variants of a target protein using LVSM 104, input sequence 106 may include a biological sequence associated with the target protein (e.g., nucleotide sequence encoding for the target protein). Determining a variant of the target protein may involve identifying a first point in the latent space of LVSM 104 corresponding to the biological sequence associated with the target protein, using the first point to identify (e.g., sample) a second point in the latent space of LVSM 104, and generating an output biological sequence associated with a first variant of the target protein using the second point. Additional biological sequences corresponding to variants of the target protein different than the first variant may be determined by identifying additional points in the latent space of LVSM 104 using the first point and LVSM 104. Accordingly, some embodiments involve identifying a third point in the latent space of LVSM 104 by using the first point, and generating, using the third point and LVSM 104, a second output biological sequence corresponding to a second variant of the target protein different than the first variant.

In some embodiments, input sequence 106 may include a human biological sequence, which may be obtained by sequencing a biological sample of a human. For example, a biological sample may be obtained from a human, and DNA may be extracted from the biological sample and sequenced to obtain the human biological sequence to use as input sequence 106. In embodiments where input sequence 106 is a human biological sequence corresponding to a target protein, using LVSM 104 to generate output sequence(s) 108 may involve sampling the latent space of LVSM 104 according to a distribution over the latent space corresponding to the human biological sequence to identify a point used to output a biological sequence associated with a variant of the target protein. Parameters of the distribution may be used in identifying the point. For example, the parameters may include a mean and a variance for each dimension of the distribution. The means may identify a point in the latent space corresponding to the human biological sequence. Identifying the point using the parameters may involve sampling the point from the latent space according to the variances. In this manner, sampling of the latent space of LVSM 104 may be considered to be near the human sequence to generate output indicating biological sequences because the distribution provides a higher probability of sampling a point proximate to a point in the latent space corresponding to the human biological sequence than a point further from the point corresponding to the human biological sequence. In some embodiments, identifying the point may include scaling the distribution by modifying one or more of the parameters to obtain a scaled distribution and sampling the point from the latent space according to the scaled distribution. The parameters may include means and variances corresponding to the human biological sequence, and sampling near the human biological sequence may involve scaling the variances by one or more factors. In instances where the distribution has multiple dimensions, different factors may be used for the variances corresponding to the different dimensions. For example, the distribution corresponding to the human biological sequence may be a five-dimensional Gaussian distribution and the five variances may be scaled by five different factors (e.g., 10, 5, 4, 2, and 0.5). Scaling the distribution may result in output sequences(s) 108 having a restricted number of mutations (e.g., amino acid substitutions) relative to the human biological sequence. According to some embodiments, an output sequence may have a number of mutations in the range of 5 to 15, or any value or range of values in that range. It should be appreciated that the one or more factors used in scaling the variances may be selected such that the output sequence(s) 108 have a desired number of mutations or average mutations.

In some embodiments, using LVSM 104 to generate output sequence(s) 108 may involve sampling the latent space of LVSM 104 within a region containing a point that corresponds to the human biological sequence to identify a point used to output a biological sequence associated with a variant of the target protein. In this manner, sampling of the latent space of LVSM 104 may be considered to be near the human sequence to generate output indicating biological sequences. In some embodiments, the region of the latent space may be identified as being within a threshold distance of the point corresponding to the human biological sequence and sampling of points corresponding to variants may be performed within the region. The threshold distance may be defined by any one or more parameters (e.g., variances) of a distribution over the latent space of LVSM 104. In some embodiments, sampling of the latent space of LVSM 104 may be constrained near a point in the latent space corresponding to a human biological sequence by variance, which may involve an amount compared to the training data.

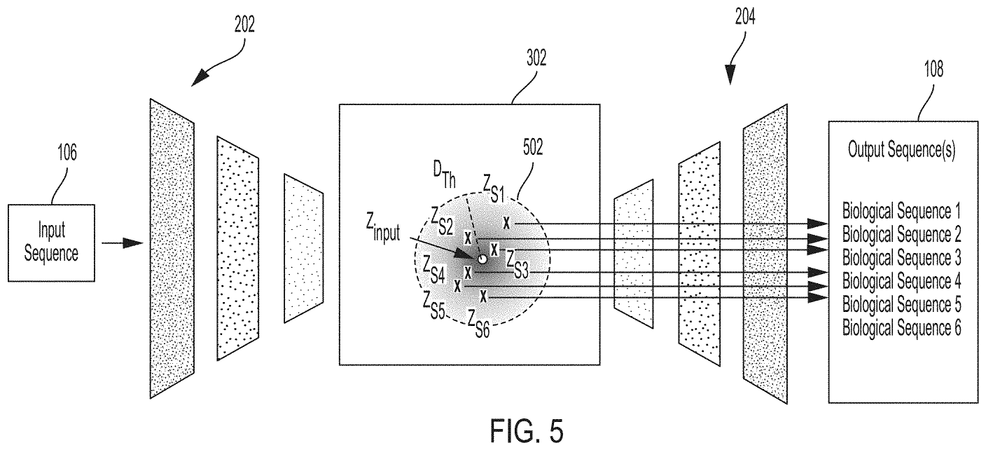

FIG. 5 is a schematic illustrating how VAE 200 may be used to generate output sequence(s) 108. In particular, input sequence 106 may be provided as an input to encoder portion 202 of VAE 200 and used to identify parameters of distribution, represented by the shading centered at point Zᵢₙₚᵤₜ, over latent space 302, such as by using encoder portion 202 to map input sequence 106 to distribution 502. Parameters of the distribution may include mean(s) and variance(s) for dimensions of the distribution. Pointᵢₙₚᵤₜ in latent space 302 may correspond to the two means of the two-dimensional distribution. The variation in the shading shown in FIG. 5 may represent probabilities of the distribution, which may depend on variances of the two-dimensional distribution. The parameters of the distribution may be used to identify sample points, including sample points Z_{S1}, Z_{S2}, Z_{S3}, Z_{S4}, Z_{S5}, and Z_{S6}, in latent space 302, such as by using one or more of the sampling techniques described herein. The sample points may be used to generate output sequence(s) 108 by using decoder portion 204 to map individual sample points in latent space 302 to respective output sequence(s) 108. For example, sample points Z_{S1}, Z_{S2}, Z_{S3}, Z_{S4}, Z_{S5}, and Z_{S6} map to Biological Sequence 1, Biological Sequence 2, Biological Sequence 3, Biological Sequence 4, Biological Sequence 5, and Biological Sequence 6, respectively. In embodiments where input sequence 106 is a biological sequence of a target protein, Biological Sequence 1, Biological Sequence 2, Biological Sequence 3, Biological Sequence 4, Biological Sequence 5, and Biological Sequence 6 may correspond to one or more variants of the target protein.

In some embodiments, point Zᵢₙₚᵤₜ may be used to identify sample points Z_{S1}, Z_{S2}, Z_{S3}, Z_{S4}, Z_{S5}, and Z_{S6} by identifying region 502 of latent space 302 containing point Zᵢₙₚᵤₜ and sampling from region 502 to determine sample points. As shown in FIG. 5, sample points Z_{S1}, Z_{S2}, Z_{S3}, Z_{S4}, Z_{S5}, and Z_{S6} are all within region 502. In some embodiments, region 502 may be identified as being within a threshold distance, D_{Th}, of point Zᵢₙₚᵤₜ. The threshold distance, D_{Th}, may be determined based on parameters of the distribution. For example, threshold distance, Dm, may be determined as being a certain number of standard deviations (e.g., 2 standard deviations) from the mean, which corresponds to point Zᵢₙₚᵤₜ. Although FIG. 5 shows region 502 as representing a circular region within latent space 302, it should be appreciated that any suitable type, shape, and size of a region in a latent space from which to sample may be implemented according to the techniques described herein. In addition, although region 502 shown in FIG. 5 has a center at point Zᵢₙₚᵤₜ, it should be appreciated that some embodiments may involve identifying a region to sample from that has a center offset from point Zᵢₙₚᵤₜ.

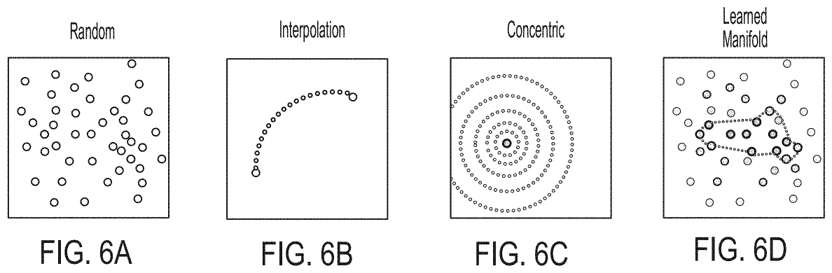

Sample points may be identified using one or more sampling techniques, including concentric sampling techniques, random sampling techniques, and interpolation sampling techniques, and learned manifold sampling techniques. FIG. 6A is a schematic of points in a latent space of a LVSM identified using a random sampling technique. FIG. 6B is a schematic illustrating how an interpolation sampling technique is performed in a latent space of a LVSM. As shown in FIG. 6B, an interpolation sampling technique may involve identifying two initial points in the latent space and determining one or more sample points along a path in latent space connecting the two initial points. According to some embodiments, initial points in the latent space may correspond to biological sequences associated with proteins having different characteristics, and using the interpolation sampling technique may involve determining a point corresponding to a biological sequence associated with a variant having both characteristics of the proteins associated with the initial points. In some embodiments, the initial points may correspond to biological sequences having biophysical and/or biochemical properties of interest. In some aspects, the initial points may be referred to as start and end points, particularly in instances where there is a directionality of the interpolation sampling process from one of the initial points (the start point) to the other initial point (the end point).

FIG. 6C is a schematic illustrating how a concentric sampling technique is performed in a latent space of a LVSM. As shown in FIG. 6C, a concentric sampling technique may involve identifying an initial point in the latent space and determining one or more sample points within and/or at the edges of regions centered on the initial point. According to some embodiments, the initial point used during concentric sampling may be a point in the latent space corresponding to a biological sequence associated with the target protein.

FIG. 6D is a schematic illustrating how a learned manifold sampling technique is performed in a latent space of a LVSM. In a learned manifold sampling technique, a region in a latent space of a LVSM may be identified by learning a manifold and sample points within the region may be identified. In some embodiments, a learned manifold sampling technique may be implemented by using a statistical model (e.g., a neural network model) for predicting a characteristic of interest for biological sequences to identify the region in the latent space to sample from. The statistical model may be trained using biological sequences, including sequences used in training the LVSM and output sequences generated by LVSM, and one or more characteristics of interest for the biological sequences, which may be obtained through experimental measurements of the biological sequences (e.g., assays for binding specificity or affinity). An output sequence generated using LVSM 104 may be passed to the statistical model to generate a prediction of the property of interest for the output sequence, which may include generating a prediction error. The statistical model may be a differentiable statistical model, which may allow for the prediction error to be back propagated, using the statistical model, to get a gradient in the latent space of the LVSM with respect to the characteristic of interest. The gradient in the latent space may then be used to identify the region in the latent space in which to sample from to determine output sequence(s) 108. In some embodiments, an iterative process of generating output sequence(s) 108 using LVSM 104, applying the statistical model to the output sequence(s) 108 to generate prediction error(s), determining a gradient in a characteristic of interest from the prediction error(s), and using the gradient to update the region in the latent space may be performed until a desired result is achieved, such as predicting the output sequence(s) from one iteration as having the characteristic of interest.

Returning to FIG. 1, output sequence(s) 108 generated using LVSM 104 may indicate multiple biological sequences associated with one or more variants of the target protein. The one or more variants may have at least the same or substantially similar activity as the target protein. In some embodiments, the one or more variants may have enhanced activity in comparison to the target protein. For example, an output sequence generated using LVSM 104 may indicate a biological sequence associated with a variant of a target RNA polymerase having a higher enzymatic activity than the target RNA polymerase.

A variant of a target protein corresponding to a biological sequence output by the LVSM may differ from the target protein at one or more residues. The number of residue sites having mutations where the variant protein has a different amino acid in comparison to the target protein may be in the range of 1-100 residues, or any number of residues within that range. In some embodiments, a variant of a target protein may have at least 30 residues with a different amino acid than the target protein. In some embodiments, a variant of a target protein may have at least 20 residues with a different amino acid than the target protein. In some embodiments, a variant of a target protein may have at least 10 residues with a different amino acid than the target protein. In some embodiments, a variant of a target protein may have at least 5 residues with a different amino acid than the target protein. A variant may have sequence similarity with the target protein for one or more conserved regions in the range of 90% to 99%, or any value or range of values in that range. In some embodiments, the variant may have at least 95% sequence similarity with the target protein for one or more conserved regions.

The techniques described herein may generate biological sequences corresponding to variants having amino acid mutations located at a variety of locations of the target protein structure, including surface sites, core sites, and boundary sites of the target protein. Accordingly, in some embodiments, a variant of the target protein determined using LVSM 104 may have a different amino acid at a surface site than the target protein. In some embodiments, a variant of the target protein determined using LVSM 104 may have a different amino acid at a core site than the target protein. In some embodiments, a variant of the target protein determined using LVSM 104 may have a different amino acid at a boundary site than the target protein.

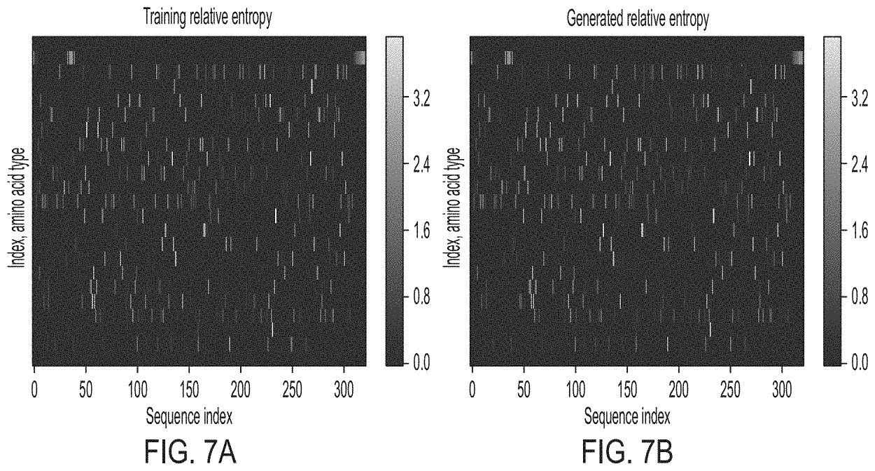

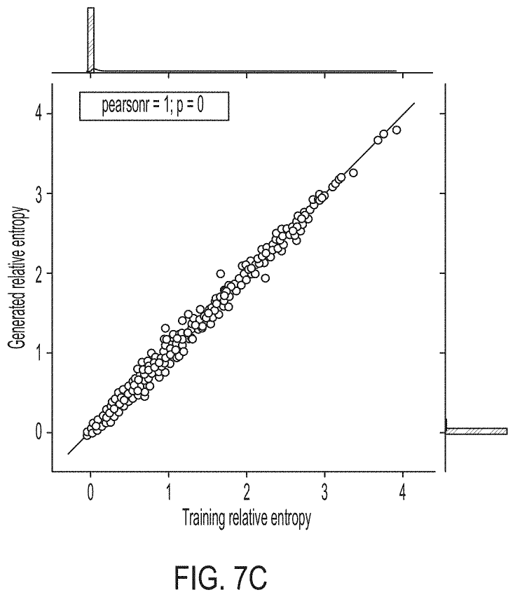

Relative entropy is one type of metric used for demonstrating the similarity between biological sequences generated using the techniques described herein and the sequences used as training data. Relative entropy provides a measurement of conservation or the amount of information in a single variable, calculated as the log ratio of the frequency that an amino acid residue appears at specific position in the aligned sequences relative to its frequency at any position in the set of known functional sequences. FIG. 7A is a plot illustrating relative entropy obtained from training sequence data. FIG. 7B is a plot illustrating relative entropy obtained from biological sequences generated from a trained LVSM using the training sequence data associated with the relative entropy shown in FIG. 7A. FIG. 7C is a plot of the relative entropy shown in FIG. 7B associated with generated biological sequences versus the relative entropy shown in FIG. 7A associated with sequences used in training the LVSM. As shown in FIG. 7C, the data has a Pearson's correlation of 1.0, demonstrating that the outputted biological sequences and the biological sequences used as training data have very similar relative entropy.

As shown in FIG. 7C, many of the residue sites of the output sequences have the same amino acid or distribution of amino acids as the sequences used as training data, indicating that the output sequences generated using LVSM 104 have regions of sequences that are conserved. In some instances, training LVSM 104 may result in LVSM 104 outputting biological sequences representative of coevolutionary relationships in the biological sequences used as the training data. The output sequences may have amino acids at particular residues that are in the training data, but the combinations of the amino acid substitutions (relative to the target protein) in a particular output sequence may be unique in comparison to the biological sequences used as training data. The amino acid substitutions may be at different residues throughout the protein structure, including the core, a boundary layer, and a surface of the protein. In some aspects, LVSM 104 may not generate output sequences that introduce an amino acid at residue that is not in one or more of the biological sequences used as training data.

The techniques described herein may configure LVSM 104 to generate output sequence(s) 108 that have similar characteristics, including pairwise relationships and higher order correlations, as the biological sequences used as training data 102. This demonstrates how the techniques described herein are effective in extracting features from training data 102 and using those features to generate novel biological sequences. Some of those features may include higher order correlations for biological sequences in training data 102, which may not otherwise be obtained using conventional protein engineering techniques. As a result, output sequence(s) 108 may have similar high order correlations as in training data 102. In particular, output sequence(s) 108 may include biological sequences that account for relationships between regions of the sequences, such as compensatory regions, in contrast to some of the conventional protein engineering techniques. Protein variants associated with such biological sequences may have improved functionality as a result of having these relationships between sequence regions over those identified using conventional techniques.

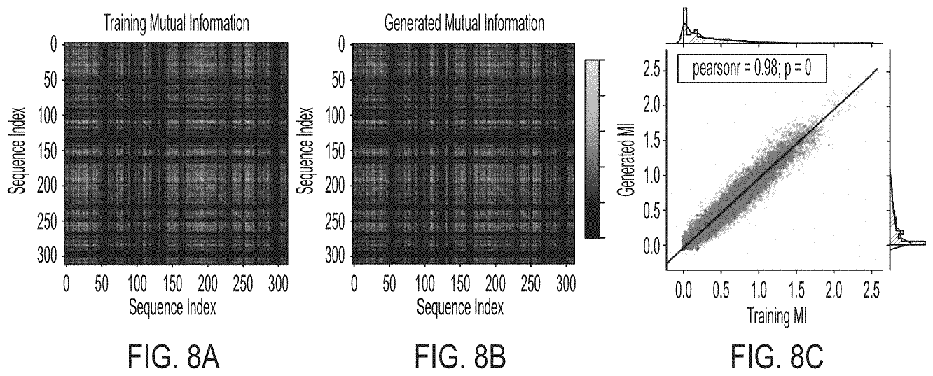

Mutual information is one type of metric used for demonstrating the similarity between biological sequences generated using the techniques described herein and the sequences used as training data. Mutual information provides a measurement in the amount of information shared between variables, which may also be considered as the entropy of the variables. FIG. 8A is a plot illustrating mutual information (e.g., pairwise statistics) obtained from training sequence data. FIG. 8B is a plot illustrating mutual information obtained from biological sequences generated from a trained LVSM using the training sequence data associated with the mutual information shown in FIG. 8A. FIG. 8C is a plot of the mutual information shown in FIG. 8B associated with generated biological sequences versus the mutual information shown in FIG. 8A associated with sequences used in training the LVSM. As shown in FIG. 8C, the data has a Pearson's correlation of 0.98, demonstrating that the outputted biological sequences and the biological sequences used as training data have similar mutual information.

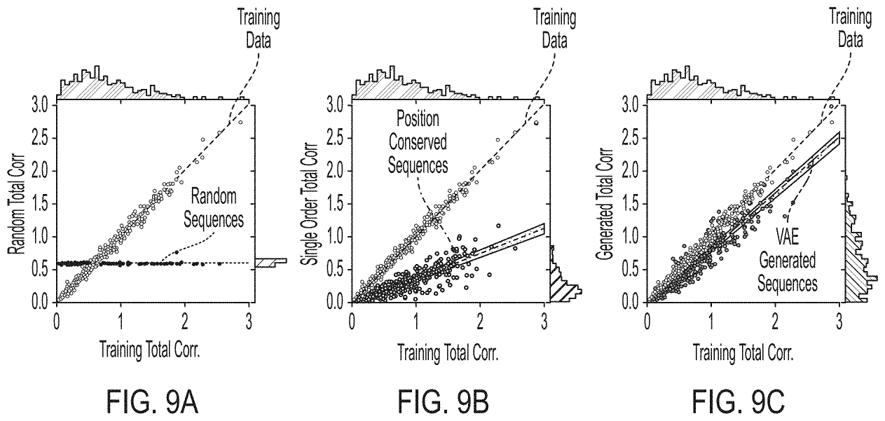

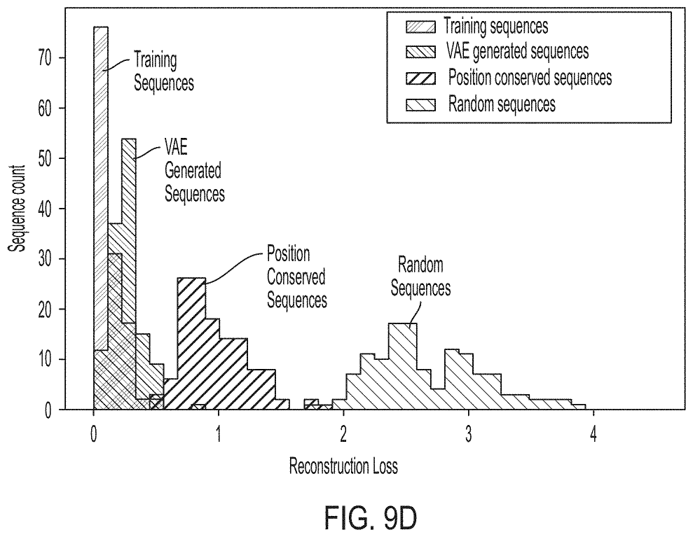

Another metric for demonstrating how output biological sequences generated using the techniques described herein are similar to the biological sequences used as training data is total correlation, which provides information on how individual variables have redundancy or dependency beyond the mutual information. FIG. 9A is a plot of total correlation for randomly generated sequences versus biological sequences used as training data. As shown in FIG. 9A, the total correlation of the randomly generated sequences is low compared to that of the training data. FIG. 9B is a plot of total correlation for position conserved biological sequences versus biological sequences used as training data. FIG. 9B shows how the total correlation of the position conserved biological sequences is higher compared to that of the randomly generated sequences, but is still low compared to the training data. FIG. 9C is a plot of total correlation for biological sequences generated using a VAE, such as VAE 200. FIG. 9C shows how the VAE generates biological sequences having a high total correlation, which is more similar to the biological sequences used as training data than the position conserved sequences. FIG. 9D is a plot of sequence count versus reconstruction loss for the training sequences, VAE generated sequences, position conserved sequences, and randomly generated sequences. FIG. 9D shows how the VAE generated sequences are most similar to the training sequences in comparison to the position conserved sequences and the randomly generated sequences.

Some embodiments may involve using sequence selection process 110 to identify selected sequence(s) 112 from among output sequence(s) 108. For example, some embodiments may involve selecting a particular variant based on one or more protein characteristics of the different variants. Sequence selection process 110 may involve determining a characteristic for individual variants, and selecting, from among output sequence(s) 108, sequence(s) 112 based on the characteristic. In some embodiments, determining the characteristic may involve identifying an amount of a protein characteristic for each of the different variants and selecting a particular variant based on the identified amounts of the protein characteristic. Examples of protein characteristics that may be used in selecting a biological sequence include protein expression level, protein half-life, protein subcellular localization, protein tissue specificity, protein immunogenicity, and protein cofactor-dependence specificity. The amounts of one or more protein characteristics may be identified using any suitable technique, including suitable protein assays and RNA-Seq analysis.

Some embodiments may involve manufacturing a biological molecule using an output biological sequence. The techniques described herein may be applied to the manufacture of different types of biological molecules, including nucleic acids and proteins, which have sequences associated with one or more variants of a target protein. As shown in FIG. 1, manufacture methods 114 may involve using selected sequence(s) 112 to manufacture biological molecule(s) 116. Manufacture methods 114 may involve any suitable techniques for synthesizing biological molecules, including polymerase chain reaction (PCR) amplification and cell transformation (e.g., bacterial transformation). In some embodiments, manufacture methods 114 may involve using an instrument for synthesizing biological molecules. In some embodiments, manufacture methods 114 may involve computer-implemented techniques, which may be performed using one or more computer hardware processors. In instances where the output biological sequence is an amino acid sequence for a variant of the target protein, computer-implemented techniques for determining a nucleotide sequence (e.g., DNA, RNA) that encodes for the amino acid sequence may be used. Such computer-implemented techniques may involve determining for at least some of the amino acids in the output biological sequence a particular codon, which includes three nucleotides that encode for a particular amino acid, based on the likelihood of that codon being present in a reference transcriptome (for RNA) or a reference genome (for DNA). In embodiments where more than one codon may encode for a particular amino acid, the codon having the highest likelihood of occurring in the reference transcriptome or reference genome may be used in determining the nucleotide sequence for the output amino acid sequence. For example, the K12 E. coli transcriptome taken from the Kazusa Codon Usage Database, may be used to determine the most common codon for particular amino acids, and those codons may be used in determining a nucleotide sequence based on an output amino acid sequence for a variation of a target protein.

Biological molecule(s) 116 may be used to produce one or more variants of the target protein. In some embodiments, biological molecule(s) 116 may be a nucleic acid (e.g., deoxyribonucleic acid (DNA), ribonucleic acid (RNA), and different types of RNA, such as messenger RNA (mRNA)) having a nucleotide sequence that encodes for a variant. In some embodiments, biological molecule(s) 116 may be a protein having an amino acid sequence corresponding to a variant determined using LVSM 104.

In some embodiments where the target protein is a human protein, manufacturing the biological molecule may involve synthesizing the biological molecule for administration to a human subject. For example, some embodiments may involve manufacturing nucleic acids (e.g., mRNA) that encode for one or more variants of the target protein and administering the nucleic acids to the human. The biological molecule may be used as a treatment for a medical condition or disease occurring in the human subject. For example, treating a medical condition or disease may involve producing, within a person's own biological cells, proteins that have the function to prevent, treat or cure the medical condition or disease. In such instances, nucleic acids (e.g., mRNA) that encode for one or more types of proteins that have such functionality, such as a variant of a target protein determined using the techniques described herein, may be used as a treatment for the medical condition or disease.

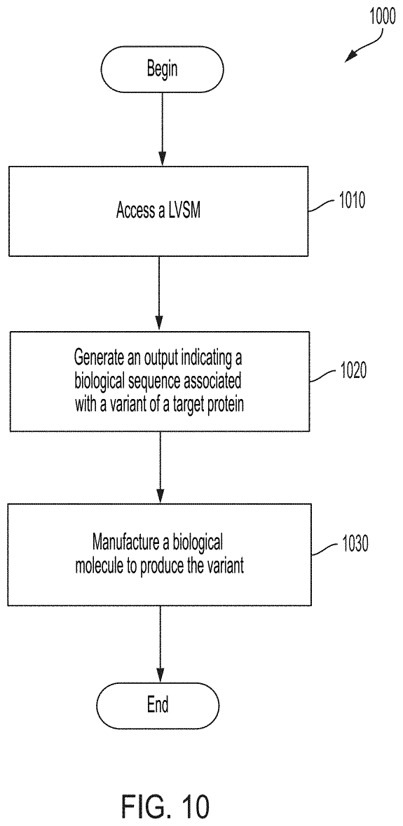

FIG. 10 is a flow chart of an illustrative process 1000 for manufacturing a variant of a protein, in accordance with some embodiments of the technology described herein. Some or all of process 1000 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, LVSM 104 and sequence selection process 110, and manufacture methods 114 may be used to perform some or all of process 1000 to manufacture a variant of a protein.

Process 1000 begins at act 1010, where a LVSM, such as LVSM 104, is accessed. The LVSM may be configured to generate output indicating one or more biological sequences corresponding to one or more variants of a target protein. Any suitable architecture may be used in the LVSM, including a multi-layer neural network, a neural network having one or more convolutional layers, and a variational autoencoder. In embodiments where the LVSM includes a variational autoencoder, the LVSM may include an encoder portion and a decoder portion. The encoder portion may be configured to map input biological sequences to distributions over the latent space of the LVSM. The decoder portion may be configured to map individual points in the latent space of the LVSM to respective output indicating a respective biological sequence corresponding to a variant of the target protein.

Some embodiments involve techniques for training the LVSM such that the LVSM may generate an output indicating one or more biological sequences corresponding to one or more variants of a target protein. In some embodiments, training the LVSM may involve using biological sequences, including a human biological sequence corresponding to the human target protein. The biological sequences may include biological sequences corresponding to the target protein occurring in organisms other than a human. The biological sequences may correspond to proteins having substantially similar functions in different species. In some embodiments, training the LVSM comprises aligning the biological sequences and using the aligned biological sequences to train the LVSM.

Next, process 1000 proceeds to act 1020, where an output indicating a biological sequence associated with a variant of a target protein is generated, such as by using LVSM 104 and sequence selection process 110. In some embodiments, an output generated from the LVSM may indicate multiple biological sequences associated with different variants of the target protein and act 1020 may further include selecting one or more biological sequences based on one or more protein characteristics of the different variants. Selecting the one or more biological sequences may involve determining a characteristic for each of the plurality of variants, and selecting, from among the plurality of biological sequences, the biological sequence associated with the target protein based on the characteristic. Examples of protein characteristics that may be used in selecting a biological sequence include protein expression level, protein half-life, protein subcellular localization, protein tissue specificity, protein immunogenicity, and protein cofactor-dependence specificity.

A variant of a target protein outputted by the LVSM may differ from the target protein at one or more residues. The number of residue sites having mutations where the variant of a target protein has a different amino acid in comparison to the target protein may be in the range of 1-100 residues, or any number or range of numbers in that range. In some embodiments, the variant of the target protein may have at least 30 residues having a different amino acid than the target protein. In some embodiments, the variant of the target protein may have at least 5 residues having a different amino acid than the target protein. In some embodiments, the variant of the target protein may have at least 95% sequence similarity with the target protein for one or more conserved regions. Different residue sites where the variant of the target protein may have one or more different amino acids than the target protein may include surface sites, core sites, and boundary sites.

Next process 1000 proceeds to act 1030, where a biological molecule to produce the variant is manufactured, such as by using manufacture methods 114. In some embodiments, manufacturing a biological molecule to produce a variant of the target protein may involve using the biological sequence. In some embodiments, the variant of the target protein may have the same or substantially similar activity as the target protein. In some embodiments, the variant of the target protein may have enhanced activity in comparison to the target protein. In some embodiments, the biological molecule includes a nucleotide sequence that encodes for the variant of the target protein. The biological molecule may be a nucleic acid, including deoxyribonucleic acid (DNA), ribonucleic acid (RNA), and different types of RNA, such as messenger RNA (mRNA). In some embodiments, the biological molecule includes an amino acid sequence associated with the variant of the target protein.

In some embodiments, the target protein is a human protein, and manufacturing the biological molecule may involve synthesizing the biological molecule for administration to a human subject. Some embodiments may further involve administering a treatment that includes the biological molecule to a human subject.

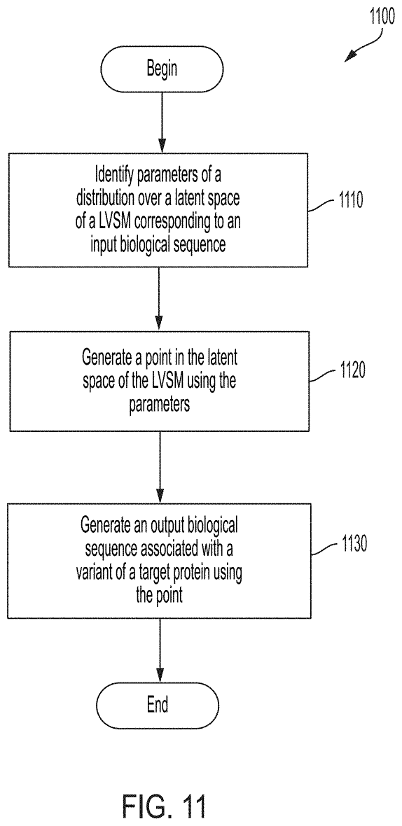

FIG. 11 is a flow chart of an illustrative process 1100 for determining a variant of a protein, in accordance with some embodiments of the technology described herein. Process 1100 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, LVSM 104 may be used to perform some or all of process 1100 to determine a variant of a protein.

Process 1100 begins at act 1110, where parameters of a distribution over a latent space of a LVSM, such as LVSM 104, corresponding to an input biological sequence is identified. Some embodiments may involve identifying the parameters of the distribution by providing the input biological sequence as input to the LVSM. In some embodiments, the LVSM is trained using biological sequences corresponding to proteins occurring in different types of organisms. In some embodiments, the biological sequences include a human biological sequence. In some embodiments, the biological sequences correspond to proteins having substantially similar functions in different species.

In some embodiments, the LVSM includes a multi-layer neural network. In some embodiments, the LVSM includes a neural network having one or more convolutional layers. In some embodiments, the LVSM includes a variational autoencoder. In such embodiments, the LVSM may include an encoder portion and a decoder portion. The encoder portion may be configured to map input biological sequences to distributions in the latent space of the LVSM. The decoder potion may be configured to map individual points in the latent space of the LVSM to respective output indicating a respective biological sequence corresponding to a variant of the target protein.

Next, process 1100 proceeds to act 1120, where a point in the latent space of the LVSM is identified using the parameters of the distribution. In some embodiments, identifying the point may involve identifying sampling the point from the latent space according to the distribution. In some embodiments, identifying the second point may involve scaling the distribution, at least in part, by modifying the parameters to obtain a scaled distribution, and sampling the point from the latent space according to the scaled distribution. In some embodiments, identifying the point involves sampling the point using a concentric sampling technique. In some embodiments, identifying the point involves sampling the point using a random sampling technique. In some embodiments, identifying the point involves sampling the point using an interpolation sampling technique. In some embodiments, identifying the point involves sampling the point using a learned manifold sampling technique.

Next, process 1100 proceeds to act 1130, where an output biological sequence associated with a variant of a target protein is generated using the point. In some embodiments, the variant has at least 30 residues having a different amino acid than the target protein. In some embodiments, the variant has at least 20 residues having a different amino acid than the target protein. In some embodiments, the variant has at least 10 residues having a different amino acid than the target protein. In some embodiments, the variant has at least 5 residues having a different amino acid than the target protein. In some embodiments, the variant has at least 95% sequence similarity with the target protein for one or more conserved regions.

In some embodiments, process 1100 may further include identifying a second point using the parameters, and generating a second output biological sequence correspond to a second variant of the target protein different from the first variant using the second point and the LVSM.

In some embodiments, process 1100 may further include manufacturing a biological molecule to produce the variant of the target protein by using the output biological sequence generated in act 1130. In some embodiments, the target protein is a human protein, and manufacturing the biological molecule may further include synthesizing the biological molecule for administration to a human subject. Some embodiments may further include administering a treatment comprising the biological molecule to the human subject.

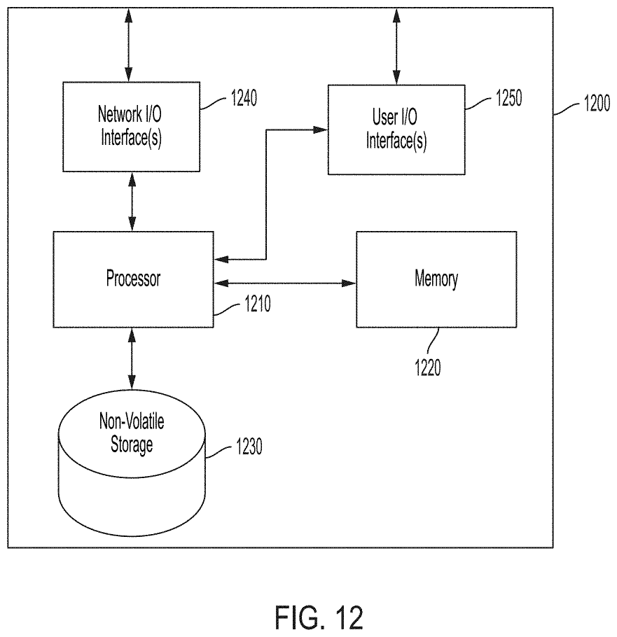

An illustrative implementation of a computer system 1200 that may be used in connection with any of the embodiments of the technology described herein is shown in FIG. 12. The computer system 1200 includes one or more processors 1210 and one or more articles of manufacture that comprise non-transitory computer-readable storage media (e.g., memory 1220 and one or more non-volatile storage media 1230). The processor 1210 may control writing data to and reading data from the memory 1220 and the non-volatile storage device 1230 in any suitable manner, as the aspects of the technology described herein are not limited in this respect. To perform any of the functionality described herein, the processor 1210 may execute one or more processor-executable instructions stored in one or more non-transitory computer-readable storage media (e.g., the memory 1220), which may serve as non-transitory computer-readable storage media storing processor-executable instructions for execution by the processor 1210.

Computing device 1200 may also include a network input/output (I/O) interface 1240 via which the computing device may communicate with other computing devices (e.g., over a network), and may also include one or more user I/O interfaces 1250, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of processor-executable instructions that can be employed to program a computer or other processor to implement various aspects of embodiments as described above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer- readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided, including with reference to FIGS. 10 and 11. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The terms “substantially,” “approximately,” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A method of manufacturing a variant of a target protein, comprising:
accessing a latent variable statistical model (LVSM) configured to generate output indicating one or more biological sequences corresponding to one or more variants of the target protein;
using the LVSM to generate a first output indicating a first biological sequence associated with a first variant of the target protein; and
manufacturing, using the first biological sequence, a first biological molecule to produce the first variant of the target protein.

2. The method of claim 1, wherein the first variant of the target protein has at least the same activity as the target protein.

3. The method of claim 1, wherein the first variant of the target protein has enhanced activity in comparison to the target protein.

4. The method of claim 1, wherein the target protein is a human protein, and manufacturing the first biological molecule further comprises synthesizing the first biological molecule for administration to a human subject.

5. The method of claim 4, further comprising:
administering a treatment comprising the first biological molecule to the human subject.

6. The method of claim 4, wherein the LVSM was trained using biological sequences including a human biological sequence corresponding to the human protein.

7. The method of claim 6, wherein the biological sequences further include biological sequences corresponding to the target protein occurring in organisms other than a human.

8. The method of claim 7, wherein the biological sequences correspond to proteins having substantially similar functions in different species.

9. The method of claim 7, wherein training the LVSM comprises aligning the biological sequences and using the aligned biological sequences to train the LVSM.

10. The method of claim 1, wherein the first variant has at least 30 residues having a different amino acid than the target protein.

11. The method of claim 1, wherein the first variant has at least 5 residues having a different amino acid than the target protein.

12. The method of claim 1, wherein the first variant has at least 95% sequence similarity with the target protein for at least one conserved region.

13. The method of claim 1, wherein a surface site of the first variant has a different amino acid than the target protein.

14. The method of claim 1, wherein a core site of the first variant has a different amino acid than the target protein.

15. The method of claim 1, wherein a boundary site of the first variant has a different amino acid than the target protein.

16. The method of claim 1, wherein the first biological molecule includes a nucleotide sequence that encodes for the first variant.

17. The method of claim 16, wherein the first biological molecule is a messenger ribonucleic acid (mRNA).

18. The method of claim 16, wherein the first biological molecule is a deoxyribonucleic acid (DNA).

19. The method of claim 1, wherein manufacturing the first biological molecule further comprises using the first biological molecule to synthesize the first variant of the target protein.

20. The method of claim 1, wherein the first biological molecule is the first variant of the target protein.

21. The method of claim 1, wherein using the LVSM further comprises:
identifying parameters of a distribution over a latent space of the LVSM corresponding to an input biological sequence obtained at least in part by sequencing a biological sample of a human;
identifying, using the parameters, a point in the latent space of the LVSM; and
identifying, using the point and the LVSM, the first biological sequence associated with the first variant of the target protein.

22. The method of claim 1, wherein the first output generated from the LVSM indicates a plurality of biological sequences associated with a respective plurality of variants of the target protein including the first variant, and the method further comprises:
determining a characteristic for each of the plurality of variants; and
selecting, from among the plurality of biological sequences, the first biological sequence based on the characteristic.

23. The method of claim 22, wherein the protein characteristic is selected from the group consisting of protein expression level, protein half-life, protein subcellular localization, protein tissue specificity, protein immunogenicity, and protein cofactor-dependence specificity.

24. The method of claim 1, wherein the LVSM includes a multi-layer neural network.

25. The method of claim 1, wherein the LVSM includes a neural network having one or more convolutional layers.

26. The method of claim 1, wherein the LVSM includes a variational autoencoder.

27. A method of determining a variant of a target protein, comprising:
identifying, for a latent variable statistical model (LVSM) configured to generate output indicating one or more biological sequences corresponding to one or more variants of the target protein, parameters of a distribution over a latent space of the LVSM corresponding to an input biological sequence obtained at least in part by sequencing a biological sample of a human;
identifying, using the parameters, a point in the latent space of the LVSM; and
identifying, using the point and the LVSM, a first output biological sequence associated with a first variant of the target protein.

28. A system comprising:
at least one hardware processor; and
at least one non-transitory computer-readable storage medium storing processor- executable instructions that, when executed by the at least one hardware processor, cause the at least one hardware processor to perform:
identifying, for a latent variable statistical model (LVSM) configured to generate output indicating one or more biological sequences corresponding to one or more variants of the target protein, parameters of a distribution over a latent space of the LVSM corresponding to an input biological sequence obtained at least in part by sequencing a biological sample of a human;
identifying, using the parameters, a point in the latent space of the LVSM; and
identifying, using the point and the LVSM, a first output biological sequence associated with a first variant of the target protein.

29. At least one non-transitory computer-readable storage medium storing processor- executable instructions that, when executed by at least one hardware processor, cause the at least one hardware processor to perform a method comprising:
identifying, for a latent variable statistical model (LVSM) configured to generate output indicating one or more biological sequences corresponding to one or more variants of the target protein, parameters of a distribution over a latent space of the LVSM corresponding to an input biological sequence obtained at least in part by sequencing a biological sample of a human;
identifying, using the parameters, a point in the latent space of the LVSM; and
identifying, using the point and the LVSM, a first output biological sequence associated with a first variant of the target protein.
